(19) United States
(12) Patent Application Publication
(10) Pub. No.: US 2013/0253392 A1
(43) Pub. Date: Sep. 26, 2013

(54) Protective Brace

(71) Applicant: Yueh-Hua Chiang, Taipei City (TW)

(72) Inventor: Yueh-Hua Chiang, Taipei City (TW)

(21) Appl. No.: 13/424,424

(22) Filed: Mar. 20, 2012

(51) Int. Cl. A61F 5/00

(52) U.S. Cl. 602/5

(57) ABSTRACT

A protective brace has a base strap, an annular pressure strap and a fastening member. The base strap includes a first end and a second end. The pressure strap is secured to the first end of the base strap and is movable through the second end of the base strap. The fastening member is movably mounted on the pressure strap and includes a fastening surface. The fastening surface faces the first end of the base strap and is fastened detachably to the pressure strap or the base strap. For a user to wear the protective brace, the base strap is first wrapped around a body part and the fastening member is then operated to make the pressure strap wrapped around the base strap and is fastened to the pressure strap or the base strap. Therefore, the protective brace can be easily worn on the user's body.

### BACKGROUND OF THE INVENTION

1. Field of the Invention

The present invention relates to a protective brace, and more particularly to a protective brace that can be easily and quickly worn on a person's body.

2. Description of the Prior Arts

A protective brace is worn on a person's body in order to protect the body from harm. With reference to FIGS. 6 and 7, a conventional protective brace comprises a base strap 50 and a pressure strap 60. The base strap 50 is made of fabric and includes an inner surface, an outer surface, and a hook component of a hook and loop fastener 51. The hook component of a hook and loop fastener 51 is attached to the inner surface of the base strap 50. The pressure strap 60 includes two ends, an outer surface, a ring 61, a loop component of a hook and loop fastener 62, a fastening portion, and a hook component of a hook and loop fastener 63. The ring 61 is connected to one end of the pressure strap 60 and is secured to the outer surface of the base strap 50. The loop component of a hook and loop fastener 62 is attached to the outer surface of the pressure strap 60. The fastening portion extends from the other end of the pressure strap 60 and has an outer surface. The hook component of a hook and loop fastener 63 is attached to the outer surface of the fastening portion.

For a user to wear the protective brace, the base strap 50 is first wrapped around a body part, such as a wrist, which is to be protected, and the hook component of a hook and loop fastener 51 of the base strap 50 is fastened to the outer surface of the fabric base strap 50. The pressure strap 60 is then wrapped around the base strap 50 to pass through the ring 61 so that the pressure strap 60 can be folded back and wrapped around the base strap 50 in reverse to make the hook component of a hook and loop fastener 63 fastened to the loop component of a hook and loop fastener 62. Thus, the protective brace is positioned on the user's body.

However, wearing the brace by operating both the base strap 50 and the pressure strap 60 requires long time and thus causes inconvenience to the user who desires to adjust the tension of the protective brace.

To overcome the shortcomings, the present invention provides a protective brace to mitigate or obviate the aforementioned problems.

### SUMMARY OF THE INVENTION

The main object of the present invention is to provide a protective brace that can be easily and quickly worn on a user's body.

To achieve the foregoing objective, the protective brace in accordance with the present invention comprises a base strap, an annular pressure strap and a fastening member. The base strap includes a first end and a second end. The pressure strap is secured to the first end of the base strap at a section and is movable through the second end of the base strap. The fastening member is movably mounted on the pressure strap and includes a fastening surface. The fastening surface faces the first end of the base strap and is fastened detachably to the pressure strap or the base strap. For a user to wear the protective brace, the base strap is first wrapped around a body part which is to be protected, and the fastening member is then operated to pull the pressure strap so as to make the first and second ends of the base strap close to each other and to make the base strap abut around the body part. The fastening member is also operated to make the pressure strap wrapped around the base strap and is fastened to the pressure strap or the base strap. Therefore, the protective brace can be easily and quickly worn on the user's body by only operating the fastening member.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a protective brace in accordance with the present invention shown in an unused condition;

FIG. 2 is a top view of the protective brace in FIG. 1 shown in an unused condition;

FIG. 3 is a perspective view of a protective brace in FIG. 1 shown in a use condition;

FIG. 4 is a top view of a protective brace in FIG. 1 shown in a use condition;

FIG. 5 is an operational perspective view of the protective brace in FIG. 1 showing that the protective brace is worn on a person's body;

FIG. 6 is a perspective view of a conventional protective brace in accordance with the prior art shown in an unused condition; and

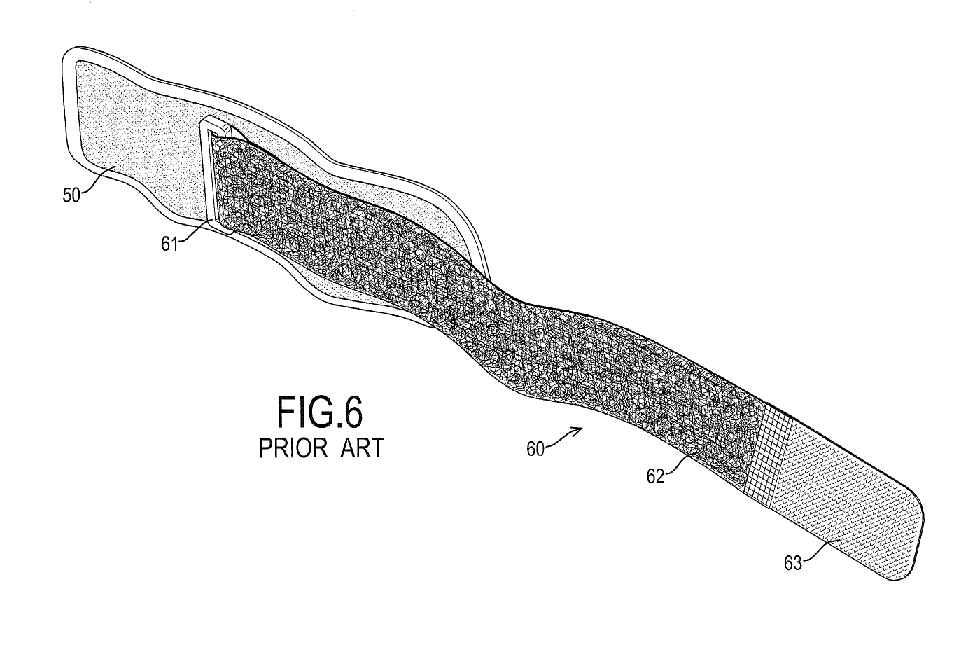

FIG. 7 is a perspective view of the conventional protective brace in FIG. 6 shown in a use condition.

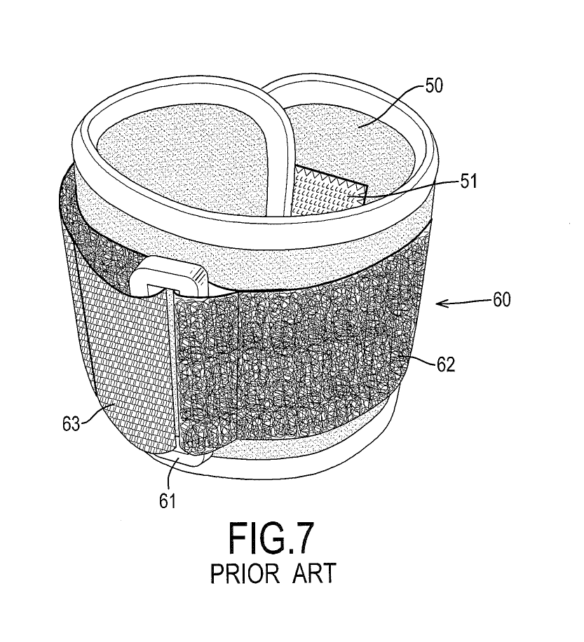

### DETAILED DESCRIPTION OF THE PREFERRED EMBODIMENTS

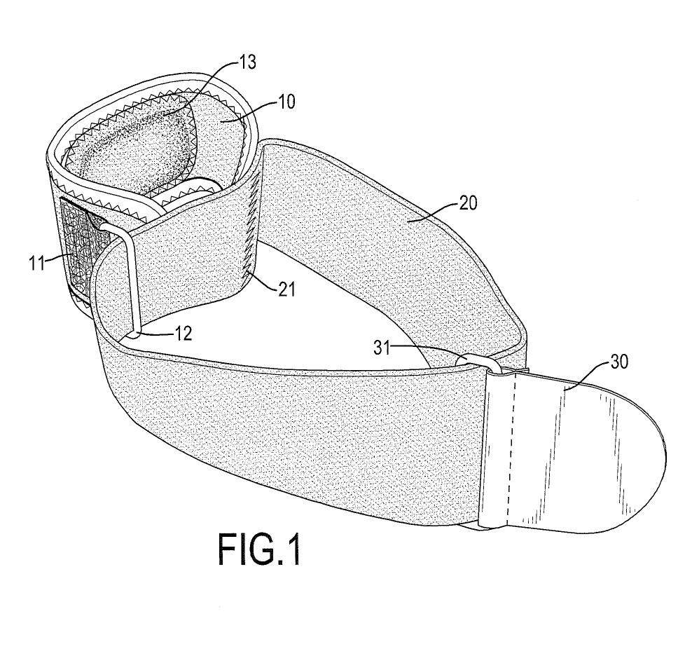

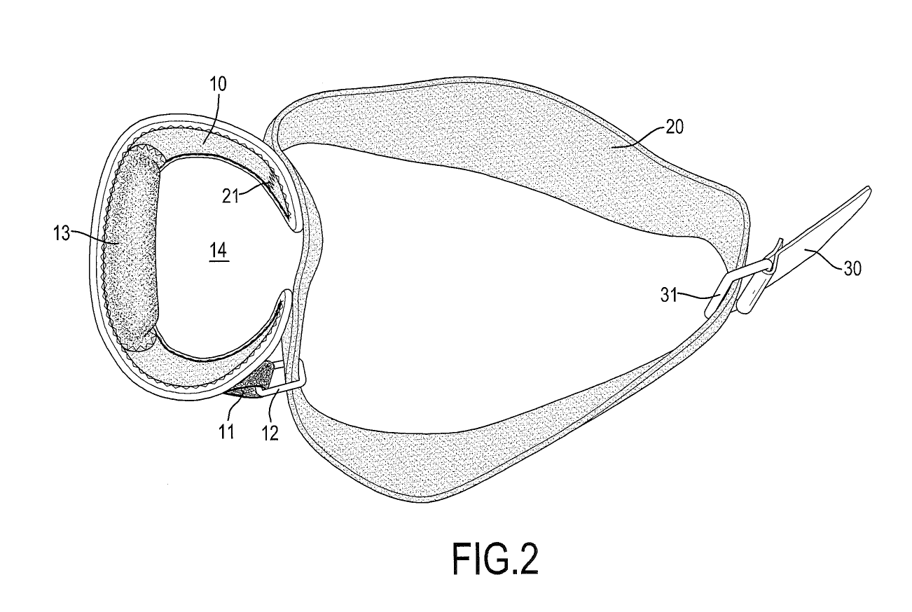

With reference to FIGS. 1 and 2, a protective brace in accordance with the present invention comprises a base strap 10, an annular pressure strap 20 and a fastening member 30.

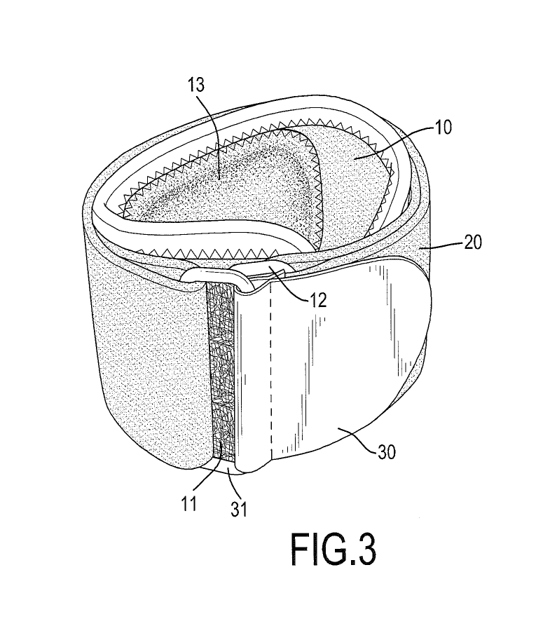

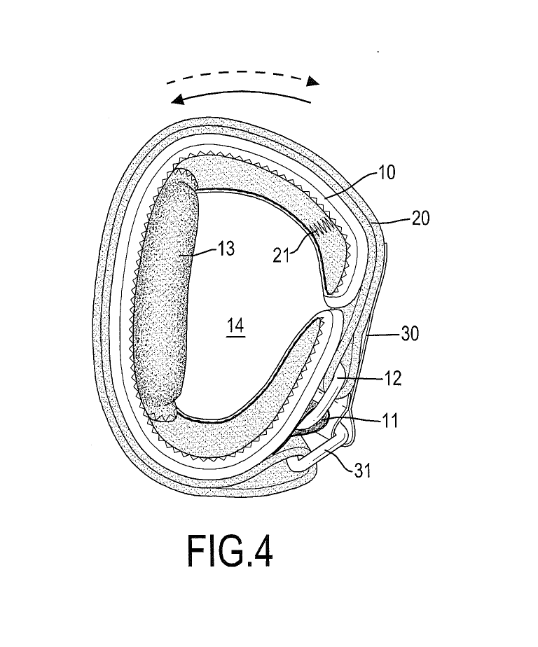

With further reference to FIGS. 3 and 4, the base strap 10 is made of fabric that allows hook components of hook and loop fasteners to be fastened thereto. The base strap 10 includes a first end, a second end, an inner surface, an outer surface, a connecting strap 11, a ring 12 and a pad 13. The connecting strap 11 is secured to the outer surface of the base strap 10 near the second end. The ring 12 is connected to the connecting strap 11. The pad 13 is attached to a middle section of the inner surface of the base strap 10.

The pressure strap 20 is made of fabric that allows hook components of hook and loop fasteners to be fastened thereto and may be elastic. The pressure strap 20 is secured to the first end of the base strap 10 at a section and is movable through the second end of the base strap 10. A space 14 is formed between the base strap 10 and the pressure strap 20. The pressure strap 20 is secured to the base strap 10 by sewing or hot pressing. In a preferred embodiment, the pressure strap 20 is sewn to the base strap 10 so as to form a sewing thread 21 and passes through the ring 12 of the base strap 10.

The fastening member 30 is movably mounted on the pressure strap 20 and includes a fastening surface. The fastening surface has a hook component of a hook and loop fastener attached thereto, faces toward the first end of the base strap 10 and is fastened detachably to the fabric pressure strap 20 or the fabric base strap 10. In a preferred embodiment, the fastening member 30 has a ring 31 connected to an end thereof and the pressure strap 20 passes through the ring 31.

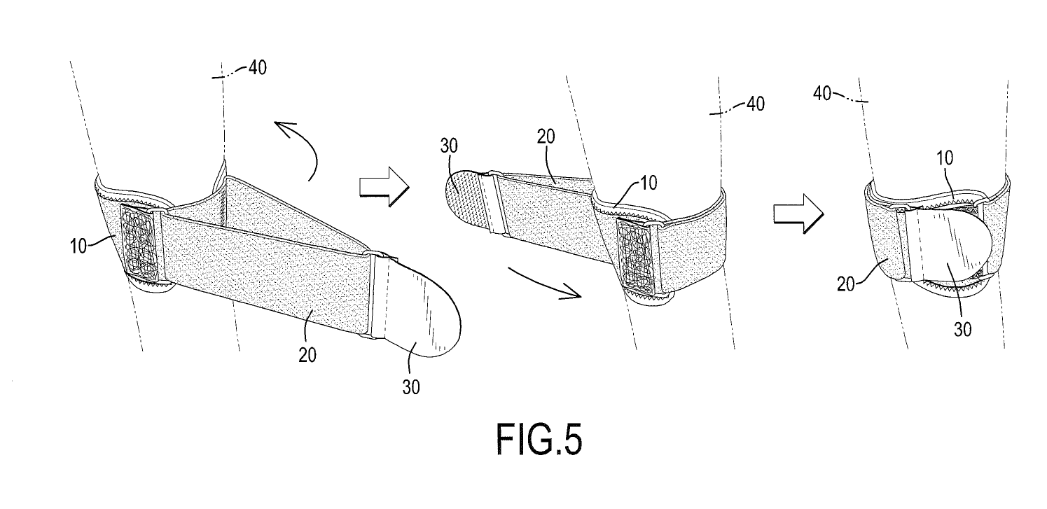

With further reference to FIG. 5, for a user to wear the protective brace of the present invention, the base strap 10 is first wrapped around a body part 40 which is to be protected to make the body part 40 placed in the space 14 between the base strap 10 and the pressure strap 20. The fastening member 30 is then operated to pull the pressure strap 20 so as to make the second end of the base strap 10 close to the first end of the base strap 10 and to make the base strap 10 abut around the body part 40. The fastening member 30 is also operated to make the pressure strap 20 wrapped around the base strap 10 in the direction as indicated by the arrows in FIG. 5 and is fastened to the fabric pressure strap 20 or the fabric base strap 10. Therefore, the protective brace can be easily and quickly worn on the user's body by only operating the fastening member 30.

In addition, with the connecting relationship of the annular pressure strap 20 and the two rings 12, 31, minor adjustments to the tension of the protective brace can be conveniently made without separating the fastening member 30 from the pressure strap 20 or the base strap 10. With reference to FIG. 4, when it is desired to tighten the protective brace, the user uses the hand to hold around the pressure strap 20 and to rotate the pressure strap 20 in the counterclockwise direction indicated by the solid line arrow in FIG. 4, the pressure strap 20 will drive the first and second ends of the base strap 10 to move toward each other, thereby improving the stability of wearing the protective brace. When it is desired to loose the protective brace, the user uses the hand to hold around the pressure strap 20 and to rotate the pressure strap 20 in the clockwise direction indicated by the broken line arrow in FIG. 4, the pressure strap 20 will drive the first and second ends of the base strap 10 to move away from each other, thereby relieving the excessive pressure from the protective brace.

Even though numerous characteristics and advantages of the present invention have been set forth in the foregoing description, together with details of the structure and features of the invention, the disclosure is illustrative only. Changes may be made in the details, especially in matters of shape, size and arrangement of parts within the principles of the invention to the full extent indicated by the broad general meaning of the terms in which the appended claims are expressed.

What is claimed is:

1. A protective brace comprising:
a base strap including a first end and a second end;
an annular pressure strap secured to the first end of the base strap at a section and being movable through the second end of the base strap; and
a fastening member movably mounted on the pressure strap and including a fastening surface facing the first end of the base strap and being fastened detachably to the pressure strap or the base strap.

2. The protective brace as claimed in claim 1, wherein
the base strap is made of fabric;
the pressure strap is made of fabric; and
the fastening surface of the fastening member has a hook component of a hook and loop fastener attached thereto and fastened detachably to the fabric pressure strap or the fabric base strap.

3. The protective brace as claimed in claim 1, wherein
the base strap includes
an outer surface;
a connecting strap secured to the outer surface of the base strap near the second end; and
a ring connected to the connecting strap; and
the pressure strap passes through the ring of the base strap.

4. The protective brace as claimed in claim 2, wherein
the base strap includes
an outer surface;
a connecting strap secured to the outer surface of the base strap near the second end; and
a ring connected to the connecting strap; and
the pressure strap passes through the ring of the base strap.

5. The protective brace as claimed in claim 1, wherein
the fastening member has a ring connected to an end thereof; and
the pressure strap passes through the ring of the fastening member.

6. The protective brace as claimed in claim 2, wherein
the fastening member has a ring connected to an end thereof; and
the pressure strap passes through the ring of the fastening member.

7. The protective brace as claimed in claim 3, wherein
the fastening member has a ring connected to an end thereof; and
the pressure strap passes through the ring of the fastening member.

8. The protective brace as claimed in claim 4, wherein
the fastening member has a ring connected to an end thereof; and
the pressure strap passes through the ring of the fastening member.

9. The protective brace as claimed in claim 1, wherein the pressure strap is sewn to the base strap.

10. The protective brace as claimed in claim 2, wherein the pressure strap is sewn to the base strap.

11. The protective brace as claimed in claim 3, wherein the pressure strap is sewn to the base strap.

12. The protective brace as claimed in claim 4, wherein the pressure strap is sewn to the base strap.

13. The protective brace as claimed in claim 5, wherein the pressure strap is sewn to the base strap.

14. The protective brace as claimed in claim 6, wherein the pressure strap is sewn to the base strap.

15. The protective brace as claimed in claim 7, wherein the pressure strap is sewn to the base strap.

16. The protective brace as claimed in claim 8, wherein the pressure strap is sewn to the base strap.

17. The protective brace as claimed in claim 13, wherein the base strap includes a pad attached to an inner surface thereof.

18. The protective brace as claimed in claim 14, wherein the base strap includes a pad attached to an inner surface thereof.

19. The protective brace as claimed in claim 15, wherein the base strap includes a pad attached to an inner surface thereof.

20. The protective brace as claimed in claim 16, wherein the base strap includes a pad attached to an inner surface thereof.
